(19) United States
(12) Patent Application Publication
(10) Pub. No.: US 2025/0174356 A1
(43) Pub. Date: May 29, 2025

(54) METHOD AND SYSTEM FOR MONITORING PATIENT ABNORMALITIES AND GENERATING RECOMMENDATIONS

(71) Applicant: Cloudphysician Healthcare Pvt Ltd, Bangalore (IN)

(72) Inventors: Dileep Raman, Bengaluru (IN); Dhruv Sud, Bengaluru (IN); Jitesh Sekar, Bengaluru (KA)

(21) Appl. No.: 18/956,098

(22) Filed: Nov. 22, 2024

(30) Foreign Application Priority Data
Nov. 23, 2023 (IN) 202341079515

(51) Int. Cl. G16H 50/20; G16H 10/60; G16H 20/10

(52) U.S. Cl. CPC G16H 50/20; G16H 10/60; G16H 20/10

(57) ABSTRACT

A method and system for monitoring patient abnormalities and generating recommendations is disclosed. In some embodiments, the method includes identifying at least one patient abnormality based on data associated with a patient and a corresponding ventilator; upon identification of the at least one patient abnormality, classifying the at least one patient abnormality into a category from a plurality of abnormality categories through a trained Machine Learning (ML) model; analyzing the classified patient abnormality based on values corresponding to a plurality of predefined parameters through the ML model; and providing recommendations to resolve the at least one patient abnormality based on the analysis.

### TECHNICAL FIELD

Generally, the invention relates to machine learning (ML). More specifically, the invention relates to a method and system for monitoring patient abnormalities and generating recommendations.

### BACKGROUND

Mechanical ventilation is a critical life-support intervention used in various healthcare settings, including Intensive Care Units (ICUs), emergency rooms, and operating rooms. Patients with respiratory insufficiency or failure rely on mechanical ventilators to maintain adequate oxygenation and carbon dioxide removal. Further, ventilator-related lung injuries may stem from abnormal interactions between a ventilator and a patient. The abnormal interactions, often referred to as “asynchronies,” not only pose a risk of lung injury but also diminish the comfort experienced by the patients undergoing mechanical ventilation. Therefore, continuous monitoring and management of patients on ventilators may be required. These complex tasks of monitoring and managing patients on mechanical ventilation require continuous assessment of various physiological parameters, including tidal volume, respiratory rate, oxygen saturation, and airway pressures among other disease patterns.

Traditionally, healthcare providers have relied on manual observations and intermittent measurements to assess patients on ventilators. However, the traditional approach has only been effective to a certain extent. The traditional approach presents several limitations such as human errors, increased staff workload, and the requirement for constant vigilance, which may result in delayed responses to critical patient events, ultimately leading to suboptimal patient outcomes.

Furthermore, various existing systems attempt to automate patient monitoring on the ventilators. However, the existing systems are limited in their ability to provide comprehensive, accurate, and timely recommendations. The existing systems may lack the capability to analyze diverse physiological data, including respiratory parameters, disease patterns, blood gases, and other vital signs, in an integrated and intelligent manner to detect and respond to patient abnormalities effectively. Further, the existing systems lack in providing explicit recommendations for corrective measures. Additionally, they are not integrated with other components of medical records.

The present invention is directed to overcome one or more limitations stated above or any other limitations associated with the known arts.

### SUMMARY

In one embodiment, a method for monitoring patient abnormalities and generating recommendations is disclosed. The method may include identifying at least one patient abnormality based on data associated with a patient and a corresponding ventilator. The method may further include classifying at least one patient abnormality into a category from a plurality of abnormality categories through a trained Machine Learning (ML) model upon identification of at least one patient abnormality. The method may further include analyzing the classified patient abnormality based on values corresponding to a plurality of predefined parameters through the ML model. The method may further include providing recommendations to resolve at least one patient abnormality based on analysis.

In another embodiment, a system for monitoring patient abnormalities and generating recommendations is disclosed. The system may include a processor and a computer-readable medium communicatively coupled to the processor. The computer-readable medium may store processor-executable instructions, which, on execution, may cause the processor to identify at least one patient abnormality based on data associated with a patient and a corresponding ventilator. The processor-executable instructions, on execution, may further cause the processor to classify the at least one patient abnormality into a category from a plurality of abnormality categories through a trained Machine Learning (ML) model, upon identification of the at least one patient abnormality. The processor-executable instructions, on execution, may further cause the processor to analyze the classified patient abnormality based on values corresponding to a plurality of predefined parameters through the ML model. The processor-executable instructions, on execution, may further cause the processor to provide recommendations to resolve at least one patient abnormality based on analysis.

In another embodiment, non-transitory computer-readable medium storing computer-executable instructions for monitoring patient abnormalities and generating recommendations is disclosed. The computer-executable instructions may be configured for identifying at least one patient abnormality based on data associated with a patient and a corresponding ventilator. The computer-executable instructions may be further configured for upon identification of the at least one patient abnormality, classifying the at least one patient abnormality into a category from a plurality of abnormality categories through a trained Machine Learning (ML) model. The computer-executable instructions may be further configured for analyzing the classified patient abnormality based on values corresponding to a plurality of predefined parameters through the ML model. The computer-executable instructions may be further configured for providing recommendations to resolve at least one patient abnormality based on analysis.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a block diagram of an exemplary system for monitoring patient abnormalities and generating recommendations, in accordance with some embodiments.

FIG. 2 illustrates a functional block diagram of a monitoring device configured for monitoring patient abnormalities and generating recommendations, in accordance with some embodiments.

FIG. 3 illustrates a flowchart of an exemplary process for monitoring patient abnormalities and generating recommendations, in accordance with some embodiments.

FIG. 4 illustrates an exemplary scenario of generating recommendations for a patient suffering from bronchospasm and supported by a ventilator, in accordance with some embodiments.

FIG. 5 illustrates a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

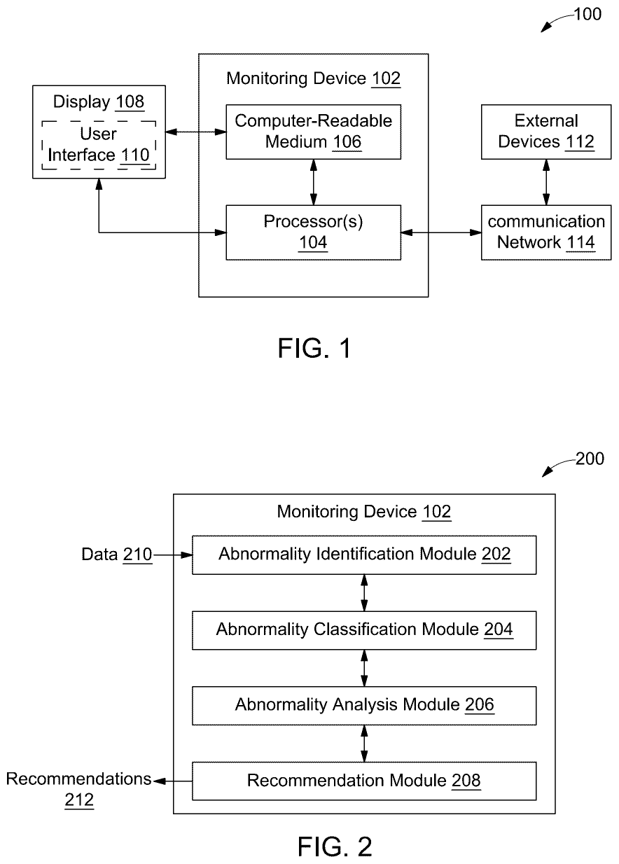

Referring now to FIG. 1, an exemplary system 100 for monitoring patient abnormalities and generating recommendations is illustrated via a block diagram, in accordance with some embodiments. The system 100 may include a monitoring device 102 (for example, a server, a desktop, a laptop, a notebook, a netbook, a tablet, a smartphone, a mobile phone, or any other computing device). The monitoring device 102 may monitor the patient abnormalities and generate the recommendations using a trained Machine Learning (ML) model. In some embodiments, the patient abnormalities may be a result of abnormal ventilator-patient interactions (i.e., asynchronies) that may reduce comfort levels of patients on mechanical ventilators. In some other embodiments, the patient abnormalities may refer to a wide range of medical conditions and symptoms that deviate from a normal or healthy state. The ML model (same as the trained ML model) may be selected from a group of a deep learning model, a Convolutional Neural Network (CNN) model, a Recurrent Neural Network (RNN) model, a Natural Language Processing (NLP) model, a Support Vector Machine (SVM), a Bayesian network, a Long Short-Term Memory (LSTM) model, an ensembled model, a Computer Vision (CV) model, a Generative Adversarial Network (GAN), an anomaly detection model, a transformer, and the like.

As will be described in greater detail in conjunction with FIGS. 2-4, the monitoring device 102 may perform various operations to monitor the patient abnormalities and generate the recommendations. For example, the monitoring device 102 identifies at least one patient abnormality based on data associated with a patient and a corresponding ventilator. Upon identification of the at least one patient abnormality, the monitoring device 102 further classifies the at least one patient abnormality into a category from a plurality of abnormality categories through the trained ML model. The monitoring device 102 further analyses the classified patient abnormality based on values corresponding to a plurality of predefined parameters through the ML model. Further, the detection device 102 provides recommendations to resolve at least one patient abnormality based on analysis. The monitoring device 102 is further explained in detail in conjunction with FIG. 2.

In some embodiments, the monitoring device 102 may include one or more processors 104 and a computer-readable medium 106 (for example, a memory). The memory may be a non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM), etc.)

Further, the computer-readable medium 106 may store instructions that, when executed by the one or more processors 104, cause the one or more processors 104 to monitor the patient abnormalities and generate the recommendations. The computer-readable medium 106 may also store various data (for example, patient vitals, an Electronic Health Record (EMR), an audio input of healthcare provider, a clinical glossary dataset, and the like) that may be captured, processed, and/or required by the system 100.

The system 100 may further include a display 108. The system 100 may interact with a user via a user interface 110 accessible via the display 108. In some embodiments, the monitoring device 102 may render results to the user via the user interface 110.

The system 100 may also include one or more external devices 112. In some embodiments, the monitoring device 102 may interact with the one or more external devices 112 over a communication network 114 for sending or receiving various data. The external devices 112 may include, but may not be limited to, a server, a desktop, a laptop, a notebook, a camera, a netbook, a ventilator, a tablet, a smartphone, a mobile phone, a remote server, a digital device, or another computing system.

In some embodiments, the monitoring device 102 and external devices 112 may communicate via the communication network 114. The communication network 114 may include, but not limited to, a vertical network, a circuit network, a Local Area Network (LAN), a Metropolitan Area Network (MAN), a Wi-fi, or the like. The communication network 114, for example, may be any wired or wireless communication network and the examples may include, but may be not limited to, the Internet, Wireless Local Area Network (WLAN), Wi-Fi, Long Term Evolution (LTE), Worldwide Interoperability for Microwave Access (WiMAX), and General Packet Radio Service (GPRS).

By way of an example, consider a patient who is on a ventilator in an Intensive Care Unit (ICU). The monitoring device 102 may continuously collect data related to the patient's vital signs and operation of the ventilator from the external devices 112 via the communication network 114. The data may include information such as heart rate, blood pressure, oxygen saturation, and ventilator settings. For instance, the monitoring device 102 tracks the patient's oxygen saturation levels and ventilator's parameters like tidal volume and respiratory rate. The monitoring device 102 may detect a sudden drop in the patient's oxygen saturation levels that may be an example of a patient abnormality. Further, the ML model, which has been trained on a large dataset, may categorize this abnormality. In this case, it categorizes the drop in oxygen saturation level as a “respiratory distress” category of abnormality, which is one of the plurality of abnormality categories. After categorizing the abnormality, the ML model further analyzes it based on the plurality of predefined parameters. For example, the monitoring device 102 may look at how severe the drop in oxygen saturation is, how long it has been occurring, and how it relates to other vital signs and ventilator settings. The ML model assesses severity and potential causes of the respiratory distress by comparing current data with expected or normal values. Based on the analysis of the classified abnormality, the monitoring device 102 may generate recommendations. In this example, the monitoring device 102 may recommend adjusting the ventilator settings, administering additional oxygen, or contacting a healthcare provider to assess the patient's condition. These recommendations may be provided to improve the patient's respiratory distress and overall well-being.

Referring now to FIG. 2, a functional block diagram 200 of the monitoring device 102 for monitoring patient abnormalities and generating recommendations is illustrated, in accordance with some embodiments. FIG. 2 is explained in conjunction with FIG. 1. The monitoring device 102 includes an abnormality identification module 202, an abnormality classification module 204, an abnormality analysis module 206, and a recommendation module 208. In some embodiments, the monitoring device 102 may include a datastore (not shown in FIG. 2) to store various data and intermediate results generated by the modules 202-208.

The abnormality identification module 202 may be configured to receive data 210 associated with a patient and a corresponding ventilator. Further, the abnormality identification module 202 may identify at least one patient abnormality based on the data 210 associated with the patient and the corresponding ventilator. The data 210 may include multimedia content displayed on a ventilator screen, an Electronic Medical Record (EMR) of the patient, patient physiology, and patient efforts. The multimedia content may be captured through at least one camera when the patient and the ventilator are in a Field of View (FoV) of at least one camera. For example, in one embodiment, the multimedia content may be captured through two different cameras.

The multimedia content may include at least one of images, a video, or an audio associated with the patient and the ventilator. In one embodiment, the multimedia content may include waveforms. For example, a ventilator screen may display graphical representations of patient data, such as respiratory waveforms, including pressure-time curves, flow-time curves, and volume-time curves. In one embodiment, the multimedia content may include alarm audio/videos. For example, if there is a critical event, the ventilator screen may display video clips showing the event, such as a patient disconnecting from the ventilator or an equipment malfunction. In one embodiment, the multimedia content may include X-radiations (X-rays) or Computed Tomography (CT) scans. For example, in some cases, X-ray or CT scan images of a patient's lungs or chest may be displayed on a monitor screen to assist in monitoring the patient's condition.

The EMR of the patient may include patient demographics including information like the patient's name, age, gender, and contact details. Further, the EMR may include a patient's medical history including details about the patient's past illnesses, surgeries, medical conditions, and the like. Moreover, the EMR may include medication records such as Information about medications the patient is currently taking, including name, dosage, route and frequency. Further, the EMR may include lab results including recent and historical data from blood tests, X-rays, and other diagnostic procedures. Also, the EMR may include physician's notes including documentation of healthcare provider's observations and assessments during patient's stay.

The patient's physiology may include vital signs including data such as heart rate, blood pressure, respiratory rate, and temperature. The patient physiology may include blood gas values (for example measurement of arterial blood gases, including oxygen and carbon dioxide levels), Electrocardiogram (ECG) data (for example, continuous monitoring of the patient's heart electrical activity), pulse oximetry (for example, monitoring of the patient's oxygen saturation levels in blood), capnography (for example, measurement of exhaled carbon dioxide levels, which indicates how well the patient is ventilating).

The patient's efforts may be analyzed through respiratory effort waveforms. The respiratory effort waveforms show a patient's inspiratory and expiratory effort, which helps assess their ability to breathe on their own. Further, to analyze the patient efforts, ventilator synchrony data may be collected. The ventilator synchrony data provides information about how well the patient is synchronized with the ventilator, which may be crucial in cases of mechanical ventilation. Further, to analyze the patient's efforts, work of breathing may be considered (for example, data related to the effort required by the patient to breathe, and the support provided by the ventilator).

By way of an example, consider a scenario where a patient is connected to a ventilator in a hospital's ICU. The patient and the ventilator may be in a FoV of camera(s) within the ICU. The camera(s) may capture multimedia content including video, images, and audio of the patient and a screen of the ventilator that may be further sent to a server associated with the monitoring device 102. In particular, the multimedia content along with an EMR of the patient, patient physiology, and patient efforts may be processed to the abnormality identification module 202. The abnormality identification module 202 may analyze this data to identify at least one patient abnormality (anomaly and critical event). For example, the patient abnormality may be “patient disconnecting from the ventilator”, or “vital signs indicating a sudden change”.

The abnormality identification module 202 may be communicatively coupled to the abnormality classification module 204. Upon identification of at least one patient abnormality, the abnormality classification module 204 may classify at least one patient abnormality into a category from a plurality of abnormality categories through a trained ML model. The plurality of abnormality categories may correspond to a plurality of predefined abnormality categories. The ML model (same as the trained ML model) may be selected from a group of a deep learning Convolutional Neural Network (CNN) model, a Recurrent Neural Network (RNN) model, a Natural Language Processing (NLP) model, a Support Vector Machine (SVM), a Bayesian network, a Long Short-Term Memory (LSTM) model, an ensemble model, a Generative Adversarial Network (GAN), a Computer Vision (CV) model, an anomaly detection model, and the like. The plurality of abnormality categories may include, but are not limited to, a ventilator synchrony abnormality, a medication dosage abnormality, a respiratory abnormality, Hypoxia or an oxygen saturation abnormality, a cardiac abnormality, a neurological abnormality, and the like. By way of an example, when the abnormality identification module 202 identifies a patient abnormality as “poor synchrony between patient's respiratory effort waveforms and ventilator's settings”. The abnormality classification module 204 may classify this abnormality into a category as “ventilator synchrony abnormality”. The abnormality classification module 204 may be communicatively coupled to the abnormality analysis module 206.

In some embodiments, the abnormality analysis module 206 may analyze the classified patient abnormality based on values corresponding to a plurality of predefined parameters through the ML model. The plurality of predefined parameters may include patient safety, patient comfort, and liberation from the ventilator. With reference to the previous example, the abnormality analysis module 206 may analyze the classified patient abnormality “ventilator synchrony abnormality” based on the plurality of predefined parameters.

By way of another example, in case an abnormality is classified as “ventilator disconnection” category, the abnormality analysis module 206 may analyze the classified patient abnormality based on the plurality of predefined parameters. For example, with regards to patient safety, the abnormality analysis module 206 may assess an extent to which ventilator disconnection poses a risk to the patient's health and well-being. Various factors such as the patient's respiratory condition, a duration of disconnection, and whether there have been any negative physiological effects may be checked. Further, with regards to the patient's comfort, the abnormality analysis module 206 may evaluate the patient's comfort during the ventilator disconnection based on a normal connection.

For example, it may analyze the abnormality to check stress or discomfort to the patient. Further, with regards to the liberation from the ventilator, the abnormality analysis module 206 may assess whether the disconnection was accidental or intentional, and if it is a sign that the patient is ready to be weaned off the ventilator. The abnormality analysis module 206 may be operatively coupled to the recommendation module 208.

In some embodiments, the recommendation module 208 may provide recommendations 212 to resolve at least one patient abnormality based on analysis. The recommendations 212 may include drug therapy and additional tests required for the patient and modifications to ventilator settings.

It should be noted that the ML model may be implemented in the monitoring device 102 to identify at least one patient abnormality, classify at least patient abnormality, analyze the classified patient abnormality, and provide the recommendations. In one embodiment, the ML model may be trained using EMRs of a plurality of the patients and the historical data corresponding to the plurality of patients and corresponding ventilators. Examples of the ML model may be, but not are limited to, an image diffusion model, a computer vision (CV) model, a forecast model, a generative AI model, and the like.

In an exemplary embodiment, let's say a premature infant is supported by a ventilator in a Neonatal Intensive Care Unit (NICU) due to respiratory distress syndrome. The abnormality identification module 202 may identify abnormalities based on data associated with the premature infant and the corresponding ventilator. Further, multimedia content (within the data) may be captured through at least one camera when the premature infant and the ventilator are in a FoV of at least one camera. The multimedia content includes at least one of an image, a video, or an audio associated with the premature infant and the ventilator. The abnormality identification module 202, in such a case, may use CV to identify the respiratory distress syndrome of the premature infant by recognizing a waveform displayed on a ventilator screen corresponding to the premature infant. The ventilator screen may show some distorted waveform corresponding to a medical condition (example, respiratory distress syndrome) of the premature infant.

Upon identification of the respiratory distress syndrome, the abnormality classification module 204 may classify the respiratory distress syndrome into a category from a plurality of abnormality categories through a trained ML model. The abnormality classification module 204 may classify the patient abnormality “respiratory distress syndrome” in a “pediatric condition” category upon analyzing the waveform of the ventilator screen by the ML model. Further, the abnormality analysis module 206 may analyze the “respiratory distress syndrome” classified as “pediatric condition” based on values corresponding to a plurality of predefined parameters through the ML model. The plurality of predefined parameters may include patient safety, patient comfort, and liberation from the ventilator. In simpler words, the abnormality analysis module 206 may analyze how safe the premature infant is in a current ventilator settings, if the premature infant is able to breathe comfortably in the current ventilator settings, and how soon the premature infant may recover or may be taken out of the ventilator support.

Further, the recommendation module 208 may provide recommendations to resolve the respiratory distress syndrome based on the analysis. The recommendations may include drug therapy and additional tests required for the premature infant, and modifications to ventilator settings. In simpler words, the recommendation module 208 may provide some prescriptive recommendations to address and manage the respiratory distress syndrome by recommending necessary interventions according to the severity of the premature infant's condition. The recommendation module 208 may also recommend taking other medical tests if the severity of the respiratory distress syndrome is not clearly identified.

It should be noted that all such aforementioned modules 202-208 may be represented as a single module or a combination of different modules. Further, as will be appreciated by those skilled in the art, each of the modules 202-208 may reside, in whole or in parts, on one device or multiple devices in communication with each other. In some embodiments, each of the modules 202-208 may be implemented as a dedicated hardware circuit comprising custom application-specific integrated circuit (ASIC) or gate arrays, off-the-shelf semiconductors such as logic chips, transistors, or other discrete components. Each of the modules 202-208 may also be implemented in a programmable hardware device such as a field programmable gate array (FPGA), programmable array logic, programmable logic device, and so forth. Alternatively, each of the modules 202-208 may be implemented in software for execution by various types of processors (e.g., processors 104). An identified module of executable code may, for instance, include one or more physical or logical blocks of computer instructions, which may, for instance, be organized as an object, procedure, function, or other construct. Nevertheless, the executables of an identified module or component need not be physically located together, but may include disparate instructions stored in different locations which, when joined logically together, include the module, and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for monitoring patient abnormalities and generating recommendations. For example, the exemplary system 100 and the associated monitoring device 102 may monitor patient abnormalities and generate recommendations by the processes discussed herein. In particular, as will be appreciated by those of ordinary skill in the art, control logic and/or automated routines for performing the techniques and steps described herein may be implemented by the system 100 and the associated monitoring device 102 either by hardware, software, or combinations of hardware and software. For example, suitable code may be accessed and executed by the one or more processors 104 on the system 100 and the associated monitoring device 102 to perform some or all of the techniques described herein. Similarly, application specific integrated circuits (ASICs) configured to perform some or all of the processes described herein may be included in the one or more processors 104 on the system 100 and the associated monitoring device 102.

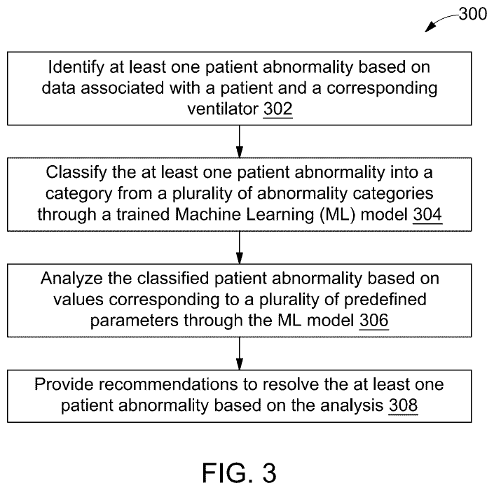

Referring now to FIG. 3, an exemplary process of a method 300 for monitoring patient abnormalities and generating recommendations is illustrated via a flowchart, in accordance with some embodiments. Each step of the process may be implemented by the monitoring device 102. FIG. 3 is explained in conjunction with the FIGS. 1-2.

At step 302, at least one patient abnormality may be identified based on data associated with a patient and a corresponding ventilator by the abnormality identification module 202. The data may include multimedia content displayed on a ventilator screen, an Electronic Medical Record (EMR) of the patient, patient physiology, and patient efforts. The multimedia content includes at least one of images, a video, or an audio associated with the patient and the ventilator. Further, the multimedia content is captured through at least one camera when the patient and the ventilator are in a Field of View (FoV) of the at least one camera.

At step 304, the at least one patient abnormality may be classified into a category from a plurality of abnormality categories through a trained Machine Learning (ML) model. This step may be performed by the abnormality classification module 204. The ML model (same as the trained ML model) may be selected from a group of a deep learning model, a Convolutional Neural Network (CNN) model, a Recurrent Neural Network (RNN) model, a Natural Language Processing (NLP) model, a Support Vector Machine (SVM), a Bayesian network, a Long Short-Term Memory (LSTM) model, an ensembled model, a Computer Vision (CV) model, a Generative Adversarial Network (GAN), an anomaly detection model, and the like.

For example, the ML model may classify the at least one patient abnormality by recognizing the pattern of the abnormality and the EMR of the patient. For example, the at least one abnormality may be classified in one of, a carotid body resection, a Cheyne-Stokes respiration, a congenital heart condition, a neuromuscular disease, chronic obstructive pulmonary disease (COPD), and the like.

At step 306, the classified patient abnormality may be analyzed based on values corresponding to a plurality of predefined parameters through the ML model. This step may be performed using the abnormality analysis module 206. The plurality of predefined parameters may include patient safety, patient comfort, and liberation from the ventilator. In some embodiments, the ML model may be trained to analyze the patient abnormality and determine a disease or changes in the ventilator setting corresponding to the patient.

At step 308, recommendations may be provided to resolve the at least one patient abnormality based on the analysis, through the recommendation module 208. The recommendations may include drug therapy and additional tests required for the patient, and modifications to ventilator settings. For example, in some embodiments, the recommendations are provided to the patient on the basis of allergic reactions, historical medical conditions, and the like.

In an exemplary embodiment, let's say a patient suffering from Acute Respiratory Distress Syndrome (ARDS) is supported by a ventilator in an ICU. When the patient is on the ventilator, a patient condition may be monitored and recommendations corresponding to the patient condition may be provided. In some embodiments, one or more patient abnormalities may be identified based on data associated with the patient and the ventilator. The data includes multimedia content displayed on a ventilator screen, an EMR of the patient, patient physiology, and patient efforts. The multimedia content may be captured through a camera when the patient and the ventilator are in a FoV of the camera. The multimedia content includes at least one of an image, a video, or an audio associated with the patient and the ventilator. In some embodiments, the ARDS may be identified as abnormality by recognizing a waveform displayed on the ventilator screen corresponding to the patient, the EMR of the patient, the patient physiology, and the patient efforts. The ventilator screen may show some distorted waveform corresponding to the medical condition of the patient.

Upon identification of the ARDS, the ARDS may be classified into a category from a plurality of abnormality categories through the ML model. It should be noted that the patient abnormality “ARDS” may be classified in a “respiratory disorder” category upon analyzing the data including the waveforms of the ventilator screen by the ML model.

Further, the “ARDS” of the “respiratory disorder” category may be analyzed based on values corresponding to a plurality of predefined parameters through the ML model. The plurality of predefined parameters may include patient safety, patient comfort, and liberation from the ventilator. In simpler words, it may be analyzed how safe patient is in the current ventilator settings, if the patient heartbeat is in comfortable range in the current ventilator settings, and how soon the patient may recover or may be taken out of ventilator support.

Further, recommendations to resolve the “ARDS” may be provided based on the analysis. The recommendations may include drug therapy and additional tests required for the patient and modifications to ventilator settings. In simpler words, recommendations to cure and keep the “ARDS” in check may be provided by recommending drugs helpful in the “ARDS”, according to the severity of the “ARDS” of the patient. Further, recommendations for taking other medical tests may also be provided if the severity of the “ARDS” is not clearly identified. In one embodiment, if the desired outcome is patient safety, changes that match the disease and physiology may be suggested and the patient safety may be prioritized as an outcome. In one embodiment, if the desired outcome is patient comfort, changes (for example, ventilator settings) may be suggested and the patient comfort may be prioritized as an outcome. In one embodiment, if the desired outcome is liberation from the ventilator, changes (for example, ventilator settings) may be suggested and the liberation from the ventilator may be prioritized as an outcome.

It should be noted that the method 300 may be implemented for a plurality of diseases and abnormalities of patients such as bronchospasm, heart diseases, polyps, cancer, brain tumors, epilepsy and other seizure disorders, and the like. However, in the disclosure, for brevity, a few examples are explained. The plurality of diseases may include, but are not limited to, respiratory diseases (abnormalities like Chronic Obstructive Pulmonary Disease (COPD), Acute Respiratory Distress Syndrome (ARDS), and bronchospasm), neuromuscular diseases (abnormalities like Amyotrophic Lateral Sclerosis (ALS), and Guillain-Barré Syndrome), Central Nervous System (CNS) disorders (abnormalities like Traumatic Brain Injury (TBI), and spinal cord injuries), cardiovascular conditions (abnormalities like Congestive Heart Failure (CHF), and cardiogenic pulmonary edema), pediatric conditions (abnormalities like prematurity and Congenital Diaphragmatic Hernia (CDH)), infectious diseases (abnormalities like severe pneumonia, and septic shock), and injury and trauma (abnormalities like chest trauma and near-drowning).

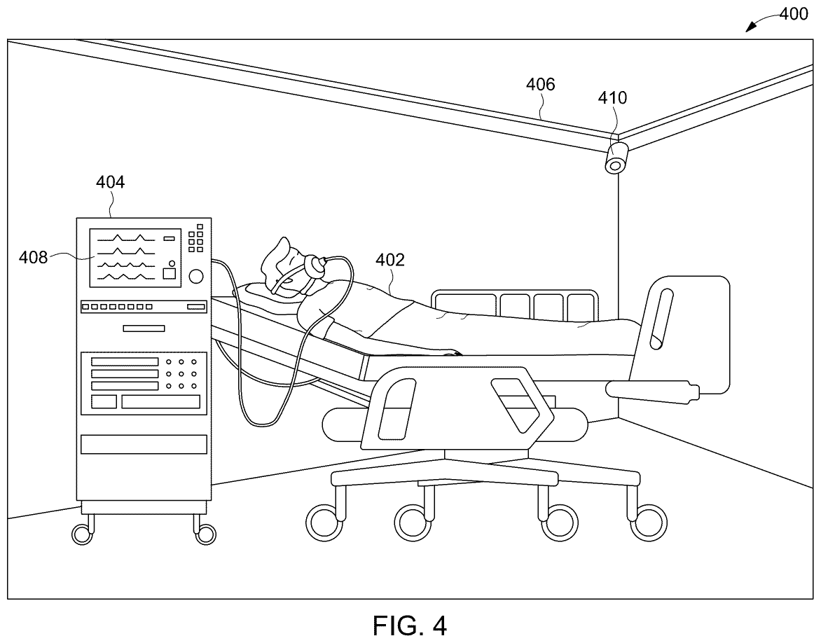

Referring now to FIG. 4, an exemplary scenario 400 of generating recommendations for a patient 402 suffering from bronchospasm and supported by a ventilator 404 in an Intensive Care Unit (ICU) 406 is illustrated, in accordance with some embodiments. FIG. 4 is explained in conjunction with FIGS. 1-3. It should be noted that the monitoring device (such as the monitoring device 102) may be used in the ICU 406 for monitoring the patient 402 and generating recommendations based on a condition of the patient 402. The monitoring device (for example, a server) may receive data associated with the patient 402 and the ventilator 404. The data may include multimedia content displayed on a ventilator screen 408, an EMR of the patient 402, patient physiology, and patient efforts.

The ICU 406 may include a camera 410. The patient 402 and the ventilator 404 may be in a Field of View (FoV) of the camera 410. The camera 410 may capture the multimedia content. The multimedia content may include at least one of images, a video, or an audio associated with the patient 402 and the ventilator 404. The monitoring device may identify one or more patient abnormalities based on the data associated with the patient 402 and the corresponding ventilator 404. In some embodiments, a Computer Vision (CV) model may be used to identify the bronchospasm of the patient 402 based on the data including a waveform displayed on the ventilator screen 408 corresponding to the patient 402. The ventilator screen 408 may show some distorted waveform corresponding to a medical condition (for example, bronchospasm) of the patient 402.

Upon identification of the bronchospasm, the monitoring device may classify the bronchospasm into a category from a plurality of abnormality categories through a trained Machine Learning (ML) model. The monitoring device may classify the patient abnormality “bronchospasm” in a “lung disease” category upon analyzing data (for example, waveforms of the ventilator screen 408 that may be received as multimedia content) through the ML model. Further, the monitoring device may analyze the classified abnormality “bronchospasm” as “lung disease” based on values corresponding to a plurality of predefined parameters through the ML model. The plurality of predefined parameters may include patient safety, patient comfort, and liberation from the ventilator. In simpler words, three things may be checked, if the patient 402 is in the current ventilator settings, if the patient 402 is able to breathe comfortably in the current ventilator settings, and how soon the patient 402 may recover or may be taken out of a ventilator support.

Further, the monitoring device may provide recommendations to resolve the bronchospasm based on the analysis. The recommendations may include drug therapy and additional tests required for the patient and modifications to ventilator settings. In simpler words, the module 208 may provide some recommendations to cure and keep the bronchospasm in check by recommending the drugs helpful in bronchospasm according to the severity of the bronchospasm (in the lung disease category) of the patient 402. For example, the monitoring device may recommend taking medical tests if severity of the bronchospasm is not clearly identified.

As will be also appreciated, the above described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

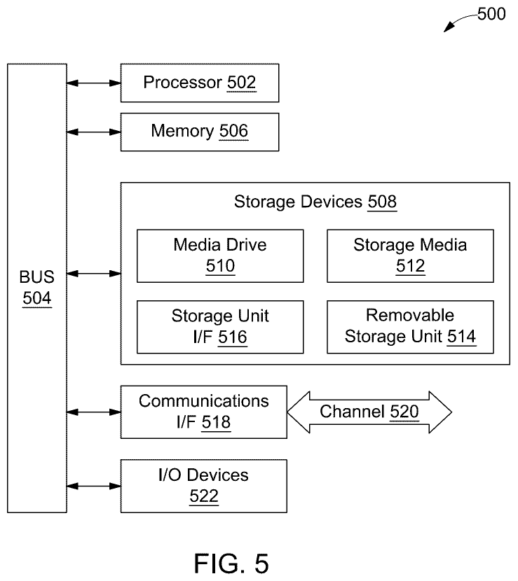

The disclosed methods and systems may be implemented on a conventional or a general-purpose computer system, such as a personal computer (PC) or server computer. Referring now to FIG. 5, an exemplary computing system 500 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 500 may represent, for example, a user device such as a desktop, a laptop, a mobile phone, personal entertainment device, DVR, and so on, or any other type of special or general-purpose computing device as may be desirable or appropriate for a given application or environment. The computing system 500 may include one or more processors, such as a processor 502 that may be implemented using a general or special purpose processing engine such as, for example, a microprocessor, microcontroller, or other control logic. In this example, the processor 502 is connected to a bus 504 or other communication medium. In some embodiments, the processor 502 may be an Artificial Intelligence (AI) processor, which may be implemented as a Tensor Processing Unit (TPU), or a graphical processor unit, or a custom programmable solution Field-Programmable Gate Array (FPGA).

The computing system 500 may also include a memory 506 (main memory), for example, Random Access Memory (RAM) or other dynamic memory, for storing information and instructions to be executed by the processor 502. The memory 506 also may be used for storing temporary variables or other intermediate information during execution of instructions to be executed by the processor 502. The computing system 500 may likewise include a read only memory (“ROM”) or other static storage device coupled to bus 504 for storing static information and instructions for the processor 502.

The computing system 500 may also include a storage device 508, which may include, for example, a media drive 510 and a removable storage interface. The media drive 510 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 512 may include, for example, a hard disk, magnetic tape, flash drive, or other fixed or removable medium that is read by and written to by the media drive 510. As these examples illustrate, the storage media 512 may include a computer-readable storage medium having stored therein particular computer software or data.

In alternative embodiments, the storage devices 508 may include other similar instrumentalities for allowing computer programs or other instructions or data to be loaded into the computing system 500. Such instrumentalities may include, for example, a removable storage unit 514 and a storage unit interface 516, such as a program cartridge and cartridge interface, a removable memory (for example, a flash memory or other removable memory module) and memory slot, and other removable storage units and interfaces that allow software and data to be transferred from the removable storage unit 514 to the computing system 500.

The computing system 500 may also include a communications interface 518. The communications interface 518 may be used to allow software and data to be transferred between the computing system 500 and external devices. Examples of the communications interface 518 may include a network interface (such as an Ethernet or other NIC card), a communications port (such as for example, a USB port, a micro USB port), Near field Communication (NFC), etc. Software and data transferred via the communications interface 518 are in the form of signals which may be electronic, electromagnetic, optical, or other signals capable of being received by the communications interface 518. These signals are provided to the communications interface 518 via a channel 520. The channel 520 may carry signals and may be implemented using a wireless medium, wire or cable, fiber optics, or other communications medium. Some examples of the channel 520 may include a phone line, a cellular phone link, an RF link, a Bluetooth link, a network interface, a local or wide area network, and other communications channels.

The computing system 500 may further include Input/Output (I/O) devices 522. Examples may include, but are not limited to a display, keypad, microphone, audio speakers, vibrating motor, LED lights, etc. The I/O devices 522 may receive input from a user and also display an output of the computation performed by the processor 502. In this document, the terms “computer program product” and “computer-readable medium” may be used generally to refer to media such as, for example, the memory 506, the storage devices 508, the removable storage unit 514, or signal(s) on the channel 520. These and other forms of computer-readable media may be involved in providing one or more sequences of one or more instructions to the processor 502 for execution. Such instructions, generally referred to as “computer program code” (which may be grouped in the form of computer programs or other groupings), when executed, enable the computing system 500 to perform features or functions of embodiments of the present invention.

In an embodiment where the elements are implemented using software, the software may be stored in a computer-readable medium and loaded into the computing system 500 using, for example, the removable storage unit 514, the media drive 510 or the communications interface 518. The control logic (in this example, software instructions or computer program code), when executed by the processor 502, causes the processor 502 to perform the functions of the invention as described herein.

Thus, the disclosed method and system try to overcome the technical problem of monitoring patient abnormalities and generating recommendations. The method and system provide means to successfully monitor patients' condition on the ventilator support with high accuracy and precision. Further, the method and system identify the patient abnormalities based on various data (for example, the ventilator settings, the multimedia content, the EMR, and the like) collected from different sources. Further, the method and system provide recommendations to the patient corresponding to the patient abnormality. Further, the method and system provide the recommendations on drug therapy, the additional tests required, and the modifications to the ventilator settings if required any. The method and system implement the Machine Learning (ML) models such as CV techniques or image diffusion model to monitor the patient abnormalities and generate the treatment recommendations.

As will be appreciated by those skilled in the art, the techniques described in the various embodiments discussed above are not routine, or conventional, or well understood in the art. The techniques discussed above provide a method and system for monitoring patient abnormalities and generating recommendations. The techniques first identify at least one patient abnormality based on data associated with a patient and a corresponding ventilator. The data includes multimedia content displayed on a ventilator screen, an Electronic Medical Record (EMR) of the patient, patient physiology, and patient efforts. The techniques then classify the at least one patient abnormality into a category from a plurality of abnormality categories through a trained Machine Learning (ML) model. The techniques then analyze the classified patient abnormality based on values corresponding to a plurality of predefined parameters through the ML model. The techniques then provide recommendations to resolve the at least one patient abnormality based on the analysis.

The disclosure provides an ability to identify and classify patient abnormalities early which can lead to timely interventions. Further, the disclosure utilizes the ML model to recognize a wide range of abnormalities, potentially before they become critical. The disclosure provides analysis of the classified abnormality based on predefined parameters allowing personalized care plans. Patients can receive tailored treatments and interventions, improving outcomes. The disclosure uses data associated with both the patient and the ventilator. Thus, the healthcare providers can make more informed decisions. This data-driven approach ensures that patient care is based on objective information. The disclosure provides an ability to recommend interventions that streamline the decision-making process for the healthcare providers. This can lead to more efficient use of resources and faster response times to patient needs. Further, the disclosure provides continuous monitoring, and reduction of the risk of oversight, ensuring that patient abnormalities are addressed promptly.

The disclosure helps enhance patient safety by reducing the likelihood of errors and improving the quality of care. The ML model may be trained to recognize various abnormality categories, making it adaptable to different medical scenarios and patient populations. Further, the disclosure may be scaled to monitor multiple patients simultaneously, making it a valuable tool in high-demand healthcare settings such as intensive care units. The healthcare providers can reduce their workload as the analysis and categorization of patient abnormalities is automated, allowing the health care providers to focus more on patient care and less on data interpretation. The disclosure early detection, analysis, and recommendations contribute to improved patient outcomes and a higher standard of care in healthcare settings.

In light of the above mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described a method and system for monitoring patient abnormalities and generating recommendations. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for monitoring patient abnormalities and generating recommendations, the method comprising:
identifying, by a monitoring device, at least one patient abnormality based on data associated with a patient and a corresponding ventilator;
upon identification of the at least one patient abnormality, classifying, by the monitoring device, the at least one patient abnormality into a category from a plurality of abnormality categories through a trained Machine Learning (ML) model;
analyzing, by the monitoring device, the classified patient abnormality based on values corresponding to a plurality of predefined parameters through the ML model; and
providing, by the monitoring device, recommendations to resolve at least one patient abnormality based on analysis.

2. The method of claim 1, wherein the data comprise multimedia content displayed on a ventilator screen, an Electronic Medical Record (EMR) of the patient, patient physiology, and patient efforts.

3. The method of claim 2, wherein the multimedia content is captured through at least one camera when the patient and the ventilator are in a Field of View (FoV) of at least one camera, and wherein the multimedia content comprises at least one of images, a video, or an audio associated with the patient and the ventilator.

4. The method of claim 1, wherein the plurality of predefined parameters comprises patient safety, patient comfort, and liberation from the ventilator.

5. The method of claim 1, wherein the recommendations comprise drug therapy and additional tests required for the patient and modifications to ventilator settings.

6. A system for monitoring patient abnormalities and generating recommendations, the system comprising:
a processor; and
a computer-readable medium communicatively coupled to the processor, wherein the computer-readable medium stores processor-executable instructions, which, on execution, cause the processor to:
identify at least one patient abnormality based on data associated with a patient and a corresponding ventilator;
upon identification of the at least one patient abnormality, classify the at least one patient abnormality into a category from a plurality of abnormality categories through a trained Machine Learning (ML) model;
analyze the classified patient abnormality based on values corresponding to a plurality of predefined parameters through the ML model; and
provide recommendations to resolve the at least one patient abnormality based on analysis.

7. The system of claim 6, wherein the data comprise multimedia content displayed on a ventilator screen, an Electronic Medical Record (EMR) of the patient, patient physiology, and patient efforts.

8. The system of claim 7, wherein the multimedia content is captured through at least one camera when the patient and the ventilator are in a Field of View (FoV) of at least one camera, and wherein the multimedia content comprises at least one of images, a video, or an audio associated with the patient and the ventilator.

9. The system of claim 6, wherein the plurality of predefined parameters comprises patient safety, patient comfort, and liberation from the ventilator.

10. The system of claim 6, wherein the recommendations comprise drug therapy and additional tests required for the patient and modifications to ventilator settings.

11. A non-transitory computer-readable medium storing computer-executable instructions for monitoring patient abnormalities and generating recommendations, the computer-executable instructions configured for:
Identifying at least one patient abnormality based on data associated with a patient and a corresponding ventilator;
upon identification of the at least one patient abnormality, classifying the at least one patient abnormality into a category from a plurality of abnormality categories through a trained Machine Learning (ML) model;
analyzing the classified patient abnormality based on values corresponding to a plurality of predefined parameters through the ML model; and
providing recommendations to resolve at least one patient abnormality based on analysis.

12. The non-transitory computer-readable medium of claim 11, wherein the data comprise multimedia content displayed on a ventilator screen, an Electronic Medical Record (EMR) of the patient, patient physiology, and patient efforts.

13. The non-transitory computer-readable medium of claim 12, wherein the multimedia content is captured through at least one camera when the patient and the ventilator are in a Field of View (FoV) of at least one camera, and wherein the multimedia content comprises at least one of images, a video, or an audio associated with the patient and the ventilator.

14. The non-transitory computer-readable medium of claim 11, wherein the plurality of predefined parameters comprises patient safety, patient comfort, and liberation from the ventilator.

15. The non-transitory computer-readable medium of claim 11, wherein the recommendations comprise drug therapy and additional tests required for the patient and modifications to ventilator settings.
